(19) United States
(12) Patent Application Publication
(10) Pub. No.: US 2022/0388949 A1
(43) Pub. Date: Dec. 8, 2022

(54) A PROCESS FOR WORKING UP WATER CONTAINING 4,4'-DICHLORODIPHENYL SULFOXIDE AND/OR 4,4'-DICHLORODIPHENYL SULFONE AS IMPURITIES

(71) Applicant: BASF SE, Ludwigshafen am Rhein (DE)

(72) Inventors: Stefan BLEI, Carl-Bosch-am Rhein (DE); Friedemann GAITZSCH, Ludwigshafen am Rhein (DE); Christian SCHUETZ, Ludwigshafen am Rhein (DE); Indre THIEL, Ludwigshafen am Rhein (DE); Petra DECKERT, Ludwigshafen am Rhein (DE); Jun GAO, Ludwigshafen am Rhein (DE); Jessica Nadine HAMANN, Ludwigshafen am Rhein (DE); Andreas MELZER, Ludwigshafen am Rhein (DE)

(21) Appl. No.: 17/775,966

(22) PCT Filed: Nov. 13, 2020
(86) PCT No.: PCT/EP2020/082063

§ 371 Date: May 11, 2022

(30) Foreign Application Priority Data
Nov. 13, 2019 (EP) 19208797.1
Apr. 1, 2020 (EP) 20167479.3

(51) Int. Cl. C07C 315/06

(52) U.S. Cl. CPC C07C 315/06

(57) ABSTRACT

The invention relates to a process for working up water containing 4,4′-dichlorodiphenyl sulfoxide and/or 4,4′-dichlorodiphenyl sulfone as impurities, comprising: (a) mixing the water containing 4,4′-dichlorodiphenyl sulfoxide and/or 4,4′-dichlorodiphenyl sulfone as impurities with an organic solvent in which 4,4′-dichlorodiphenyl sulfoxide and/or 4,4′-dichlorodiphenyl sulfone have a solubility of at least 0.5 wt % based on the amount of 4,4′-dichlorodiphenyl sulfoxide and/or 4, 4′-dichlorodiphenyl sulfone and organic solvent at 20° C., which forms a two-phase system with water and which can be stripped from water with a stripping gas and subsequently separating the obtained mixture into an aqueous phase and an organic phase, and (b) stripping the organic solvent from the aqueous phase with a stripping gas.

A process for working up water containing 4,4′-dichlorodiphenyl sulfoxide and/or 4,4′-dichlorodi-phenyl sulfone as impurities

### Description

The invention relates to a process for working up water containing 4,4′-dichlorodiphenyl sulfox-ide and/or 4,4′-dichlorodiphenyl sulfone as impurities.

Water containing 4,4′-dichlorodiphenyl sulfoxide (in the following DCDPSO) and/or 4,4′-dichloro-diphenyl sulfone (in the following DCDPS) as impurities may originate for example from a pro-cess for producing DCDPSO and/or a process for producing DCDPS. Further, water containing DCDPS and/or DCDPSO may originate from processes which use DCDPS and or DCDPSO for example as a monomer for preparing polymers like polyarylene(ether)sulfones such as polyether sulfone or polysulfone or as an intermediate of pharmaceuticals, dyes or pesticides.

Processes for the production of DCDPSO and DCDPS are known. Thus, DCDPSO can be obtained for instance by a Friedel-Crafts reaction from thionyl chloride and chlorobenzene in the presence of aluminum chloride and subsequent hydrolysis. Oxidation of DCDPSO can lead to DCDPS, for example as described in WO-A 2018/007481 and SU-A 765262. Further processes for producing DCDPS are described for example in U.S. Pat. Nos. 4,937,387, 5,082,973, WO-A 2011/131508, WO-A 2016/201039 or WO-A 2011/067649.

Generally, in all of these processes, water which contains impurities accrues e.g. during the hydrolysis and during oxidization of the DCDPSO. These impurities for example comprise DCDPSO and/or DCDPS and the reactants used for the process, particularly monochlorobenzene.

Therefore, it is an object of the present invention to provide a process for working up water containing DCDPSO and/or DCDPS. Particularly, it is an object to provide a process with which water with low or very low amounts of impurities is obtained, such as a low TOC content. In particular, a process was aimed at which can provide working up water from a process for the manu-facture of DCDPSO and/or DCDPS or which comprises the use of DCDPSO and/or DCDPS.

Thereby also a process was sought which is efficient with respect to energy consumption and removal of impurities. Further it is an object to provide a process for working up water in which valuable products which are contained in the water are regained.

This object is achieved by a process for working up water containing DCDPSO and/or DCDPS as impurities, comprising:
(a) mixing the water containing DCDPSO and/or DCDPS as impurities with an organic solvent in which DCDPSO and/or DCDPS have a solubility of at least 0.5 wt % based on the amount of DCDPSO and/or DCDPS and organic solvent at 20° C., which forms a two-phase system with water and which can be stripped from water with a stripping gas and subsequently separating the obtained mixture into an aqueous phase and an organic phase,
(b) stripping the organic solvent from the aqueous phase with a stripping gas.

The water containing DCDPSO and/or DCDPS as impurities is in the following also termed as “polluted water”. The aqueous phase obtained by phase separation in (a) in the following also is termed as “water depleted in DCDPSO and/or DCDPS”.

The organic solvent in which DCDPSO and/or DCDPS have a solubility of at least 0.5 wt % based on the amount of DCDPSO and/or DCDPS and organic solvent at 20° C., and which forms a two-phase system with water and which can be stripped from water with a stripping gas is in the following also termed as “organic solvent”.

The solubility of DCDPSO and/or DCDPS in the organic solvent can be determined as

$$S = \frac{{m}_{D}}{{m}_{s o l v} + {m}_{D}} \cdot 100 [ wt \% ] with$$

$${m}_{D} = amount of DCDPSO and / or DCDPS in kg$$

$${m}_{s o l v} = amount of solvent in kg$$

A two-phase system which the organic solvent and the water form is each system in which the amount of organic solvent and the amount of water used in the process form two liquid phases at the respective process conditions at which the phase separation is carried out. Two liquid phases are formed, if the organic solvent and the water have a miscibility gap. According to IU-PAC, Compendium of Chemical Technology, 2^{nd} edition “Gold Book”, Version 2.3.3, 2014-02-24, page 937, the miscibility gap can be represented by a phase diagram, in which the composition of a mixture of two different liquids is plotted against the temperature. Connecting the points gives the bimodal curve. The bimodal curve encloses the area in which two liquids build out two phases, thus these phases are separated. Outside the bimodal curve the liquids are miscible, thus form a homogenous mixture.

An organic solvent which can be stripped from water is each organic solvent which has a vapor pressure in the aqueous phase being higher than the partial pressure of the organic solvent in the stripping gas.

### In the Present Application

any description, even though described in relation to a specific embodiment, is applicable to and interchangeable with other embodiments of the present disclosure;
where an element or component is said to be included in and/or selected from a list of recited elements or components, it should be understood that in related embodiments explicitly contemplated here, the element or component can also be any one of the individual recited elements or components, or can also be selected from a group consisting of any two or more of the explicitly listed elements or components; any element or component recited in a list of elements or components may be omitted from such list; and
any recitation herein of numerical ranges by endpoints includes all numbers subsumed within the recited ranges as well as the endpoints of the range and equivalents.

A person of ordinary skill in the art will recognize additional ranges within the explicitly disclosed ranges are contemplated and within the scope of the present disclosure.

By mixing the polluted water with the organic solvent and the following phase separation, the DCDPSO and/or the DCDPS which is transferred into the organic phase by this process also can be obtained as product in the process and thereby the yield of DCDPSO and/or DCDPS can be increased.

Preferably, the process comprises using organic solvent in which DCDPSO and/or DCDPS have a solubility in the range from 0.5 to 80 wt % at 20° C. and particularly of 3 to 50 wt % at 20° C.

Further, it is preferred, that the solubility of DCDPSO and/or DCDPS at the boiling point of the organic solvent is up to 100 wt % in each case based on the amount of DCDPSO and/or DCDPS and organic solvent.

The organic solvent with which the polluted water is mixed in (a) can be one organic solvent or a mixture of two or more organic solvents. Usually under the aspect of less process complexity and in particular for industrial set-ups it is preferred that only one organic solvent is used. The organic solvent preferably is one of chlorobenzene, pentane, hexane, cyclohexane, methyl cyclohexane, heptane, octane, toluene, xylene, ethylbenzene, 1-hexanol, 1-octanol, diethylketone, 2-hexanone, propyl acetate, butyl acetate, hexanoic acid, heptanoic acid or a mixture of at least two of these. If the polluted water originates from a process for producing DCDPSO and/or a process for producing DCDPS, it is preferred that the organic solvent with which the polluted water is mixed in (a) is the same organic solvent as used in the process for producing DCDPSO and/or the process for producing DCDPS. Particularly preferably, the organic solvent is chlorobenzene, particularly monochlorobenzene.

Particularly for polluted water originating from a process for producing DCDPSO which therefore contains DCDPSO as impurity, using the same organic solvent as used in the process for producing DCDSO, particularly using monochlorobenzene, has the further advantage, that the DCDPSO enriched organic solvent obtained as the organic phase can be recycled into the pro-cess for producing DCDPSO, either into the reaction or in the work-up process of the crude reaction product. Further, using the same organic solvent as used in the process for producing DCDSO, particularly using chlorobenzene, in the process has the additional advantage that precipitation of solidified DCDPSO can be avoided.

For water comprising DCDPS, it is also possible to recycle the organic solvent into the process if the same organic solvent is used as in the process for producing DCDPSO. Also in this case, the organic solvent preferably is recycled into the process for producing DCDPSO. By recycling water comprising DCDPS into the process for producing DCDPSO, the DCDPS passes the process without being damaged and thus the product yield of the complete process producing DCDPSO and using the DCDPSO for production of DCDPS can be increased. Alternatively, particularly if it is not possible to recycle the organic solvent into a process for producing DCDPSO, the organic solvent after being used for working up the polluted water is incinerated.

By stripping the organic solvent from the aqueous phase with the stripping gas, the organic solvent can be regained, at least partially separated from the stripping gas for example by phase separation or in a distillation process and if the same organic solvent is used in the production process of DCDPSO reused in the production process of the DCDPSO. If only a partial separation takes place, for example in a distillation process, organic solvent and a part of the water are removed at the top of the distillation column. This mixture forms a two-phase condensate and may be used as such in a washing step in the process for producing DCDPSO.

Besides DCDPSO and/or DCDPS, the polluted water may further comprise at least one of chlorobenzene, hydrogen chloride (HCl), alkali metal sulfates like sodium sulfate (Na₂SO₄), metal chlorides, particularly aluminum chloride (AlCl₃), carboxylic acids or alcohols, particularly methanol, ethanol or toluene as impurities. The impurities contained in the polluted water thereby de-pend on the process the polluted water originates from. It is a particular advantage of the inventive process that it can be used for removing DCDPS and/or DCDPSO from polluted water which contains only very small amounts thereof. Thus the process is particularly suitable for working-up polluted water which comprises from 0.5 ppm to 3000 ppm DCDPSO and/or 0.5 ppm to 3 wt % DCDPS and 0 ppm to 10 wt % chlorobenzene besides further impurities.

If the polluted water contains hydrogen chloride or a metal chloride, these usually remain in the aqueous phase which is subjected to further water purifying processes, particularly neutralization, for example in a water purification plant. Alcohols or carboxylic acids which may be com-prised as impurities in the polluted water usually disperse into the aqueous phase and the organic phase. That part which remains in the aqueous phase also may be removed by further water purifying processes as carried out in a water purification plant. That part which migrates into the organic phase may remain in the organic phase even if the organic phase is recycled as it usually has no detrimental effect on the production process of DCDPSO and DCDPS.

If the polluted water comprises DCDPSO, the polluted water for example originates from a process for producing DCDPSO comprising:
(i) reacting thionyl chloride, chlorobenzene and aluminum chloride in a molar ratio of thionyl chloride: chlorobenzene: aluminum chloride of 1: (6 to 9): (1 to 1.5) at a temperature in the range from 0 to below 20° C., forming an intermediate reaction product and hydrogen chloride,
(ii) mixing aqueous hydrochloric acid and the intermediate reaction product at a temperature in the range from 70 to 110° C. to obtain a crude reaction product comprising DCDPSO, (iii) separating the crude reaction product into an organic phase comprising the DCDPSO and an aqueous phase,
(iv) washing the organic phase with an extraction liquid.

To obtain DCDPSO, in the reaction (i) thionyl chloride, chlorobenzene and aluminum chloride are fed into a reactor in a molar ratio of thionyl chloride: chlorobenzene: aluminum chloride of 1 : (6 to 9): (1 to 1.5), preferably in a molar ratio of thionyl chloride: chlorobenzene: aluminum chloride of 1: (7 to 9): (1 to 1.2) and particularly in a molar ratio of thionyl chloride: chlorobenzene: aluminum chloride of 1: (7 to 8): (1 to 1.1).

The thionyl chloride and the chlorobenzene react in the presence of the aluminum chloride whereby an intermediate reaction product and hydrogen chloride form. The intermediate reaction product comprises 4,4′-dichlorodiphenyl sulfoxide-AlCl₃ adduct. The aluminum chloride generally can act as catalyst.

The hydrogen chloride (HCl) produced in the reaction typically is in gaseous form and at least partly removed from the reactor. While it can be put to other use in gaseous form, preferably, the hydrogen chloride removed from the reaction is mixed with water to produce aqueous hydrochloric acid.

After the reaction the intermediate reaction product is mixed with aqueous hydrochloric acid. For reasons of energy as well as production efficiency as well as sustainability, particularly preferably, the aqueous hydrochloric acid is produced from the hydrogen chloride removed from the reaction (i). By mixing the intermediate reaction product with the aqueous hydrochloric acid hydrolysis of the intermediate reaction product can take place. A crude reaction product comprising DCDPSO is obtained. The crude reaction product can also comprise aluminum chloride which is typically in hydrated form, usually as AlCl₃·6H₂O.

After finishing the hydrolysis, the mixture separates into an aqueous phase comprising the AlCl₃ and an organic phase comprising DCDPSO solved in the excess chlorobenzene. In case the mixture is stirred, stirring is stopped to allow the mixture to separate.

The amount of aqueous hydrochloric acid used in (ii) preferably is such that no aluminum chloride precipitates and that further two liquid phases are formed the lower phase being the aqueous phase and the organic phase being the upper phase. To achieve this, the amount of aqueous hydrochloric acid used in (ii) preferably is such that after the hydrolysis the weight ratio of aqueous to organic phase is in the range from 0.6 to 1.5 kg/kg, more preferably in the range from 0.7 to 1.0 kg/kg and particularly in the range from 0.8 to 1.0 kg/kg is obtained. A smaller amount of aqueous hydrochloric acid may result in precipitation of aluminum chloride. Particularly at higher concentrations of the aqueous hydrochloric acid a larger amount is necessary to avoid precipitation. Therefore, the concentration of the aqueous hydrochloric acid preferably is kept below 12 wt %.

To remove the aqueous hydrochloric acid and remainders of the aluminum chloride from the organic phase, the organic phase obtained in (iii) is separated off and washed with an extraction liquid.

After being separated off, the organic phase is fed into the washing step (iv) to remove residual aluminum chloride and hydrochloric acid. The extraction liquid used for washing the organic phase preferably is water.

The washing preferably is carried out at a temperature in the range from 70 to 110° C., more preferred in a range from 80 to 100° C. and particularly in a range from 80 to 90° C. Particularly preferably the washing is carried out at the same temperature as the hydrolysis.

Generally, the amount of extraction liquid which preferably is water is sufficient to remove all or essentially all of the aluminum chloride from the organic phase. Under the aspect of waste con-trol it is usually preferred to use as little extraction liquid as possible. The amount of water used for washing preferably is chosen in such a way that a weight ratio of aqueous to organic phase in the range from 0.3 to 1.2 kg/kg, more preferably in the range from 0.4 to 0.9 kg/kg and particularly in the range from 0.5 to 0.8 kg/kg is obtained. In terms of sustainability and avoidance of large waste water streams it is preferred to use as little water for the washing step as possible.

It is particularly preferred to use such an amount of water, that the entire aqueous phase from the washing step can be used to generate the aqueous hydrochloric acid in the concentration needed for hydrolysis.

After a predetermined washing period, mixing is stopped to allow the mixture to separate into an aqueous phase and an organic phase. The aqueous phase and the organic phase are removed from the washing vessel separately. The organic phase comprises the DCDPSO solved in the excess chlorobenzene as solvent. The predetermined washing period preferably is as short as possible to allow for short overall process times. At the same time, it needs sufficient time to allow for the removal of aluminum chloride.

The process may comprise one or more than one such washing cycles. Usually one washing cycle is sufficient.

The DCDPSO can be separated off the organic phase according to any process known to a skilled person. The organic phase for example can be cooled down to allow the DCDPSO to crystallize.

The polluted water to be worked-up in the inventive process may comprise the aqueous phase obtained in (iii) and the water which is used as extraction liquid in the washing (iv). However, it is particularly preferred to use the water which is used as extraction liquid in the washing (iv) for producing the aqueous hydrogen chloride which is used in the reaction (ii). Thus, only the aqueous phase obtained in (iii) is the polluted water to be worked-up in the inventive process. Using the water which was used as extraction liquid in the washing (iv) for producing the aqueous hydrogen chloride has the additional advantage that the total amount of water to be removed from the process as waste water can be reduced.

The polluted water obtained in the production process of DCDPSO usually comprises 0.01 to 3 wt % chlorobenzene, 1 to 12 wt % hydrogen chloride, 10 to 30 wt % AlCl₃ and 50 to 3000 ppm DCDPSO. More preferred, the amount of chlorobenzene in the polluted water is in the range from 0.02 to 0.5 wt %, the amount of hydrogen chloride in the range from 3 to 12 wt %, the amount of AlCl₃ in a range from 15 to 30 wt % and the amount of DCDPSO in the range from 100 to 2000 ppm. Particularly, the amount of chlorobenzene in the polluted water is in the range from 0.05 to 0.3 wt %, the amount of hydrogen chloride in the range from 8 to 11 wt %, the amount of AlCl₃ in a range from 17 to 25 wt % and the amount of DCDPSO in the range from 200 to 1500 ppm. All amounts in wt % and ppm are based on the total amount of polluted water.

If the polluted water comprises DCDPS as impurity, the polluted water particularly may originate from a process for producing DCDPS by oxidizing DCDPSO, comprising:
(1) reacting DCDPSO and an oxidizing agent in a carboxylic acid as solvent to obtain a reaction mixture comprising DCDPS and carboxylic acid;
(II) separating the reaction mixture comprising DCDPS and carboxylic acid into a residual moisture comprising DCDPS as crude product and a liquid phase comprising carboxylic acid, and
(III) optionally working up the residual moisture comprising DCDPS.

The DCDPSO used in (1) preferably originates from the process for producing DCDPSO described above. As the presence of chlorobenzene in the reaction (1) may result in formation of an explosive gas phase or liquid phase and in toxic by-products, it is preferred to wash the DCDPSO with a carboxylic acid before feeding the DCDPSO into the reaction (1). By this washing, remainders of the chlorobenzene are removed.

The carboxylic acid used for washing the DCDPSO and the carboxylic acid used as solvent in (1) preferably is the same. The carboxylic acid thereby can be only one carboxylic acid or a mixture of at least two different carboxylic acids. Preferably the carboxylic acid is at least one aliphatic carboxylic acid. The at least one aliphatic carboxylic acid may be at least one linear or at least one branched aliphatic carboxylic acid or it may be a mixture of one or more linear and one or more branched aliphatic carboxylic acids. Preferably the aliphatic carboxylic acid is an aliphatic C₆ to C₁₀ carboxylic acid, particularly a C₆ to C₉ carboxylic acid, whereby it is particularly preferred that the at least one carboxylic acid is an aliphatic monocarboxylic acid. Thus, the at least one carboxylic acid may be hexanoic acid, heptanoic acid, octanoic acid nonanoic acid or decanoic acid or a mixture of one or more of said acids. For instance the at least one carboxylic acid may be n-hexanoic acid, 2-methyl-pentanoic acid, 3-methyl-pentanoic acid, 4-methyl-pentanoic acid, n-heptanoic acid, 2-methyl-hexanoic acid, 3-methyl-hexanoic acid, 4-methyl-hexanoic acid, 5-methyl-hexanoic acid, 2-ethyl-pentanoic acid, 3-ethyl-pentanoic acid, n-octanoic acid, 2-methyl-heptanoic acid, 3-methyl-heptanoic acid, 4-methyl-heptanoic acid, 5-methyl-heptanoic acid, 6-methyl-heptanoic acid, 2-ethyl-hexanoic acid, 4-ethyl-hexanoic acid, 2-propyl pentanoic acid, 2,5-dimethylhexanoic acid, 5,5-dimethyl-hexanoic acid, n-nonanoic acid, 2-ethyl-hepatnoic acid, n-decanoic acid, 2-ethyl-octanoic acid, 3-ethyl-ocantoic acid, 4-ethyl-octanoic acid. The carboxylic acid may also be a mixture of different structural isomers of one of said acids. For instance, the at least one carboxylic acid may be isononanoic acid comprising a mixture of 3,3,5-trimethyl-hexanoic acid, 2,5,5-trimethyl-hexanoic acid and 7-methyl-octanoic acid or neodecanoic acid comprising a mixture of 7,7-dimethyloctanoic acid, 2,2,3,5-tetramethyl-hexanoic acid, 2,4-dimethyl-2-isopropylpentanoic acid and 2,5-dimethyl-2-ethylhexanoic acid.

Particularly preferably, the carboxylic acid is n-hexanoic acid or n-heptanoic acid.

The reaction (1) of DCDPSO and the oxidizing agent in a carboxylic acid as solvent in principle can be operated as known by a skilled person from WO-A 2018/007481.

It is preferred that in the reaction (1) for producing DCDPS using the DCDPSO, generally a solution is used comprising DCDPSO and carboxylic acid in a weight ratio of DCDPSO to carboxylic acid in a range from 1: 2 to 1: 6, more preferred in a range from 1: 2 to 1: 4 and particularly in a range from 1: 2,5 to 1: 3,5. By such a ratio of DCDPSO to carboxylic acid, the solubility of DCDPS produced by oxidation of the DCDPSO is at an optimum at the temperature of the oxidization reaction and of a subsequent crystallization process for obtaining crystallized DCDPS.

Such a ratio particularly allows for a sufficient heat dissipation in the reaction and an amount of DCDPS in the mother liquor obtained by crystallization which is as low as possible.

Usually the reaction (1) can be carried out at elevated temperature, particularly at a temperature in the range from 70 to 110° C.

To obtain DCDPS, the solution comprising DCDPSO and carboxylic acid is oxidized by an oxidizing agent. Therefore, the oxidizing agent preferably is added to the solution to obtain a reaction mixture. From the reaction mixture the residual moisture comprising DCDPS can be obtained.

The oxidizing agent used for oxidizing DCDPSO for obtaining DCDPS preferably is at least one peroxide. The at least one peroxide may be at least one peracid, for example one or a mixture of two or more, such as three or more peracids. Preferably, the reaction (VI) is carried out in the presence of one or two, particularly in the presence of one peracid. The at least one peracid may be a linear or branched C₁ to C₁₀ peracid, which may be unsubstituted or substituted, e.g. by linear or branched C₁ to C₅ alkyl or halogen, such as fluorine. Examples thereof are peracetic acid, performic acid, perpropionic acid, percaprionic acid, pervaleric acid or pertrifluoroacetic acid. Particularly preferably the at least one peracid is a C₆ to C_{I1} peracid, for example 2-ethyl-hexanoic peracid. If the at least one peracid is soluble in water, it is advantageous to add the at least one peracid as aqueous solution. Further, if the at least one peracid is not sufficiently soluble in water, it is advantageous that the at least one peracid is dissolved in the respective carboxylic acid. Most preferably, the at least one peracid is a linear or branched C₆ to C_{I1} peracid which is generated in situ. Particularly preferably, the peracid is generated in situ by using hydrogen peroxide (H₂O₂) as oxidizing agent.

In the oxidizing process, particularly when using H₂O₂ as oxidizing agent, water is formed. Further, water may be added with the oxidizing agent. Preferably, the concentration of the water in the reaction mixture is kept below 5 wt %, more preferred below 3 wt % and particularly below 2 wt %. By using aqueous hydrogen peroxide with a concentration of 70 to 85 wt % the concentration of water during the oxidization reaction is kept low. It even may be possible to keep the concentration of water in the reaction mixture during the oxidization reaction below 5 wt % without removing water by using aqueous hydrogen peroxide with a concentration of 70 to 85 wt %.

Additionally or alternatively, it may be necessary to remove water from the process for keeping the concentration of water in the reaction mixture below 5 wt %. To remove the water from the process, it is for example possible to strip water from the reaction mixture. Stripping thereby preferably is carried out by using an inert gas as stripping medium. If the concentration of water in the reaction mixture remains below 5 wt % when using aqueous hydrogen peroxide with a concentration of 70 to 85 wt % it is not necessary to additionally strip water. However, even in this case it is possible to strip water to further reduce the concentration.

To obtain the DCDPS as product, the reaction mixture is separated into a residual moisture comprising DCDPS (in the following also termed as “moist DCDPS”) and a liquid phase comprising the carboxylic acid in (II).

If the moisture in the moist DCDPS does not have a negative effect on processes which use the DCDPS, the moist DCDPS can be withdrawn from the process as a crude product. However, it is preferred to further work-up the moist DCDPS.

The separation can be carried out by any known process, for example by a distillation or by cooling to form a suspension and subsequent solid-liquid separation of the suspension. Particularly preferably, the reaction mixture is separated by cooling and subsequent solid-liquid separation.

Preferably, for separating the reaction mixture into the moist DCDPS and the liquid phase comprising the carboxylic acid, the reaction mixture is cooled to a temperature below the saturation point of DCDPS to obtain a suspension comprising crystallized DCDPS and a liquid phase. The suspension is separated by a solid-liquid separation into moist DCDPS and a second mother liquor. The solid-liquid separation thereby can be carried out by any suitable separation means for example by filtration or centrifugation.

The cooling for crystallizing DCDPS can be carried out in any crystallization apparatus or any other apparatus which allows cooling of the organic mixture, for example an apparatus with surfaces that can be cooled such as a vessel or tank with cooling jacket, cooling coils or cooled baffles like so-called “power baffles”.

Cooling of the reaction mixture for crystallization of the DCDPS can be performed either continuously or batchwise. To avoid precipitation and fouling on cooled surfaces, it is particularly preferred that separating the reaction mixture in (II) comprises:
(II.a) mixing the reaction mixture with water in a gastight closed vessel to obtain a liquid mixture;
(II.b) cooling the liquid mixture obtained in (Vll.a) to a temperature below the saturation point of DCDPS by
   reducing the pressure in the gastight closed vessel to a pressure at which the water starts to evaporate,
   condensing the evaporated water by cooling
   mixing the condensed water into the liquid mixture in the gastight closed vessel,
to obtain a suspension comprising crystallized DCDPS;
(II.c) carrying out a solid-liquid-separation of the suspension to obtain the moist DCDPS and the liquid phase comprising the carboxylic acid.

This process allows for cooling the DCDPS comprising reaction mixture without cooling surfaces onto which particularly at starting the cooling process crystallized DCDPS accumulates and forms a solid layer. This enhances the efficiency of the cooling process. Also, additional efforts to remove this solid layer can be avoided.

If cooling is performed according to (II.b), the suspension which is subjected to the solid-liquid separation additionally contains water besides the crystallized DCDPS and the carboxylic acid.

After completing the cooling and crystallization by pressure reduction, the process is finished and preferably the pressure is set to ambient pressure, again. After reaching ambient pressure, the suspension which formed by cooling the liquid mixture in the gastight closed vessel is subjected to the solid-liquid separation (II.c). In the solid liquid separation process, the crystallized DCDPS formed by cooling is separated from the carboxylic acid and the water.

To purify the moist DCDPS, the moist DCDPS preferably is washed with an aqueous base in a first phase and subsequently with water in a second phase. By washing, particularly remainders of the carboxylic acid and further impurities, for example undesired by-products which formed during the process for producing the DCDPS are removed.

To reduce the amount of carboxylic acid which is withdrawn from the process and disposed, the aqueous base is mixed with the strong acid after being used for washing.

The aqueous base used for washing the moist DCDPS can be one aqueous base or a mixture of at least two aqueous bases. Preferably, the aqueous base used for washing in the first phase preferably is an aqueous alkali metal hydroxide or a mixture of at least two aqueous alkali metal hydroxides, for example aqueous potassium hydroxide or sodium hydroxide, particularly sodium hydroxide.

By using the aqueous alkali metal hydroxide, the anion of the carboxylic acid reacts with the al-kali metal cation of the alkali metal hydroxide forming an organic salt and water. In difference to the carboxylic acid which generally is not soluble in water and depending on the carboxylic acid also even may be immiscible with water, the organic salt formed by reaction with the aqueous base is soluble in water and thus remainders which are not removed with the aqueous alkali metal hydroxide and the water formed by the reaction can be removed from the moist DCDPS by washing with water. This allows to achieve DCDPS as product which contains less than 1 wt %, preferably less than 0.7 wt % and particularly less than 0.5 wt % organic impurities.

As the water of the aqueous base and the water produced by the reaction of the anion of the base with the carboxylic acid generally is not sufficient to remove all of the organic salt and as further part of the aqueous base may stay in the moist DCDPS, the moist DCDPS is washed with water in the second phase. By washing with water, remainders of the organic salt and of the aqueous base which did not react are removed. The water then can be easily removed from the DCDPS by usual drying processes known to a skilled person to obtain dry DCDPS as product. Alternatively, it is possible to use the water wet DCDPS which is obtained after washing with water in subsequent process steps.

The washing with water in the second phase preferably is carried out in two washing steps. In this case, it is particularly preferred to use fresh water for the washing in the second washing step and to use the water which has been used in the second washing step in the first washing step. This allows the amount of water which is used for washing in total to be kept low.

The polluted water which is worked up according to the inventive process may be the water stripped from the reaction mixture, the water used for cooling and crystallization and separated off in the solid-liquid separation and finally the water used for washing the moist DCDPS. The polluted water obtained in the separate process steps can be worked-up separately or combined. Preferably, the polluted water of all process steps of the production of DCDPS are mixed and then worked up combined. Independently of being mixed and worked up combined or worked up separately, the polluted water obtained in the production process of DCDPS is water comprising DCDPS as impurity. However, particularly preferably, the polluted waters withdrawn from the DCDPS production are mixed and worked up combined. The combined polluted waters withdrawn from the DCDPS production usually comprise 1 ppm to 3 wt % DCDPS, 1 ppm to 10 wt % carboxylic acid, 1 ppm to 20 wt % alcohols, particularly methanol or ethanol, 0 ppm to 5 wt % alkali metal salts and 0 ppm to 5 wt % metal chlorides, more preferred 1 ppm to 2 wt % DCDPS, 1 ppm to 5 wt % carboxylic acid, 1 ppm to 15 wt % alcohols, particularly methanol or ethanol, 1 ppm to 2 wt % alkali metal salts and 1 ppm to 3 wt % metal chlorides and particularly 2 ppm to 0.5 wt % DCDPS, 1 ppm to 2 wt % carboxylic acid, 0.05 wt % to 10 wt % alcohols, particularly methanol or ethanol, 1 ppm to 1 wt % alkali metal salts and 1 ppm to 1 wt % metal chlorides, all amounts being based on the total mass of the polluted water.

Water comprising DCDPSO and DCDPS particularly is a mixture of the polluted waters obtained in a process for producing DCDPSO and a process for producing DCDPS by oxidizing DCDPSO. In this case the polluted waters obtained in the processes are not worked up separately but together in one process.

If the polluted waters originating from the process for producing DCDPSO and the process for producing DCDPS are mixed and worked up combined, the polluted water usually comprises 1 ppm to 1000 ppm DCDPSO, 1 ppm to 3 wt % DCDPS, 1 ppm to 3 wt % chlorobenzene, 1 ppm to 12 wt % hydrogen chloride, 1 ppm to 30 wt % metal chloride, particularly AlCl₃, 1 ppm to 10 wt % carboxylic acid, 1 ppm to 20 wt % alcohols, particularly methanol or ethanol, and 1 ppm to 15 wt % alkali metal salts, particularly Na₂SO₄. More preferred, the polluted water comprises 1 ppm to 500 ppm DCDPSO, 1 ppm to 2 wt % DCDPS, 1 ppm to 0.5 wt % chlorobenzene, 1 ppm to 5 wt % hydrogen chloride, 1 ppm to 25 wt % metal chloride, particularly AlCl₃, 1 ppm to 5 wt % carboxylic acid, 1 ppm to 10 wt % alcohols, particularly methanol or ethanol, and 1 ppm to 10 wt % alkali metal salts, particularly Na₂SO₄, and particularly, the polluted water usually comprises 1 ppm to 300 ppm DCDPSO, 1 ppm to 1 wt % DCDPS, 1 ppm to 0.2 wt % chlorobenzene, 1 ppm to 3 wt % hydrogen chloride, 1 ppm to 15 wt % metal chloride, particularly AlCl₃, 1 ppm to 3 wt % carboxylic acid, 1 ppm to 5 wt % alcohols, particularly methanol or ethanol, and 1 ppm to 5 wt % alkali metal salts, particularly Na₂SO₄. All amounts in wt % and ppm are based on the total amount of polluted water.

Particularly if the polluted water is withdrawn from different process steps, it is preferred, to collect the polluted water in a buffer container from which the polluted water is fed into the process for working up the polluted water. On the other hand, the process for working up the polluted water also may be configured such that all of the polluted water which is withdrawn from the production process of DCDPSO is fed directly into the mixing with the organic solvent and the subsequent phase separation (a). If the polluted water is obtained in different process steps, in this case it is preferred to mix the polluted water in a suitable mixing unit before mixing with the organic solvent in (a). By mixing the polluted water withdrawn from different process steps or by collecting the polluted water in a buffer container before feeding into the mixing step (a), variations in the composition of the polluted water which may result in variations in the composition of the different streams obtained by the process for working up the polluted water can be pre-vented. Such variations in the composition of the different streams obtained in the process for working up the polluted water particularly may have the effect that due to the variations the streams cannot be recycled into the process for producing DCDPSO as they would have a negative effect on the process for producing DCDPSO and thus also on the product quality.

Due to the different composition of the polluted waters obtained in the process for producing DCDPSO and the polluted waters obtained in the process for producing DCDPS, it may be preferred to work-up the polluted waters obtained in the process for producing DCDPSO and the polluted waters obtained in the process for producing DCDPS separately. In this case, it is preferred, to mix the polluted waters obtained in the process for producing DCDPSO and work them up and to mix the polluted waters obtained in the process for producing DCDPS and work them up separately from the polluted waters obtained in the process for producing DCDPSO.

Besides for purifying polluted water obtained in a process for producing DCDPS according to the process as described above, the inventive process for working up polluted water containing DCDPSO and/or DCDPS also can be used for working up the polluted water which accrues in alternative processes for producing DCDPS.

The inventive process for example can be used for working up polluted water which is obtained in a process for producing DCDPS by reacting chlorobenzene and sulfur trioxide as described for example in U.S. Pat. No. 4,937,387.

In this process, in a first reaction stage liquid sulfur trioxide reacts with chlorobenzene, forming 4-chlorobenzenesulfonic acid. In the first reaction stage, the chlorobenzene is added in excess and the first reaction stage usually is operated at a temperature in a range between −20° C. and 230° C., preferably in a range between 30 and 70° C. Further, it is preferred to add water to the first reaction stage. In a second stage which preferably is operated at a temperature in a range between 180 and 250° C., DCDPS is formed by reaction of the 4-chlorobenzenesulfonic acid with chlorobenzene. For removing water which also is formed in the second stage, superheated vaporous chlorobenzene is used as a stripping medium. Further, the superheated vaporous chlorobenzene is used for heating.

For removing remainders of the 4-chlorobenzenesulfonic acid, the reaction mixture obtained in the second reaction stage is subjected to an extraction process with water. The 4-chlorobenzenesulfonic acid obtained in the water is dried and recycled into the second reaction stage.

The organic phase which contains the DCDPS may be subject to further steps for working up, for example by crystallization.

Polluted water obtained by this process usually may comprise 1 ppm to 10 wt % monochlorobenzene, 1 ppm to 50 wt % 4-chlorobenzenesulfonic acid and 0.5 ppm to 1 wt % DCDPS. Further, the polluted water additionally may contain 0 to 30 wt % sulfuric acid or sulfates. A further process for producing DCDPS in which water containing DCDPS as impurity may accrue, comprises a reaction stage in which sulfur trioxide, dimethyl sulfate and chlorobenzene are reacted in a single reaction at a temperature in the from 50 to 100° C. Such a process for example is described in U.S. Pat. No. 5,082,973. Besides reacting sulfur trioxide, dimethyl sulfate and chlorobenzene in a single reaction, it is also possible to carry out a first reaction stage in which sulfur trioxide and dimethyl sulfate are converted to dimethyl pyrosulfate and in a second reaction stage the dimethyl pyrosulfate reacts with chlorobenzene forming DCDPS. The obtained reaction product is washed with water and then dried.

If the reaction is carried out in two stages, it is preferred that in the second stage the dimethyl pyrosulfate is dosed into a reactor which contains chlorobenzene and after finishing adding the dimethyl pyrosulfate, the resulting reaction mixture is transferred into a vessel containing a mixture comprising chlorobenzene and water, the mixture of chlorobenzene and water having a temperature in the range from 50 to 100° C. In the vessel a suspension forms which is filtrated.

By filtration solid DCDPS is obtained and a two phasic filtrate, comprising an aqueous phase and an organic phase.

Polluted water obtained by this process usually may comprise 1 ppm to 10 wt % monochlorobenzene, 1 ppm to 50 wt % 4-chlorobenzenesulfonic acid, 0.5 ppm to 1 wt % DCDPS, 0 to 15 wt % methanol or toluene and 1 ppm to 25 wt % dimethyl sulfate. Further, the polluted water additionally may contain 0 to 30 wt % sulfuric acid or further sulfates.

Independently of the process which is carried out for producing DCDPS, each aqueous phase which accrues in the process and may contain DCDPS can be subjected to the inventive process for working up water comprising DCDPSO and/or DCDPS. If in a process polluted water accrues in different stages, it is possible to mix the polluted waters or to work up the polluted water of each stage separately.

Besides for polluted water obtained in process for producing DCDPS, the process for working up polluted water also may be used for working up polluted water obtained in processes which use DCDPS and/or DCDPSO for example as a monomer for preparing polymers like poly-arylene(ether)sulfones such as polyether sulfone or polysulfone or as an intermediate of pharmaceuticals, dyes and pesticides, for example processes for producing diamino-diphenylsulfone which may be used as antimicrobiological substance or as a drug. Further processes which may use DCDPS and/or DCDPSO in which water which contains DCDPS may accrue, for example are processes for producing insecticides, processes in rubber manufacturing and processes for producing epoxy systems.

To regain the DCDPSO and/or the DCDPS from the polluted water and thus increase the yield of the respective production process, the polluted water is mixed with the organic solvent and subsequently subjected to a phase separation in (a). By this process the DCDPSO and/or the DCDPS is separated off the polluted water by an extraction using the organic solvent as extraction liquid. By this extraction, the water depleted in DCDPSO and/or DCDPS and the organic phase comprising organic solvent and DCDPSO and/or DCDPS are obtained.

The organic phase comprising organic solvent and DCDPSO and/or DCDPS which is withdrawn from the mixing with the organic solvent and the subsequent phase separation (a) preferably is recycled into the production process of DCDPSO. Alternatively, the organic phase comprising organic solvent and DCDPSO and/or DCDPS, particularly an organic phase comprising organic solvent and DCDPS can be disposed or incinerated externally. Preferably, the organic phase comprising organic solvent and DCDPSO and/or DCDPS is recycled into the hydrolysis which is carried out in the organic solvent. Alternatively, it is also possible to recycle the organic phase comprising organic solvent and DCDPSO and/or DCDPS into the washing (iv) or the separating step (iii). Preferably, the organic phase comprising organic solvent and DCDPSO and/or DCDPS is recycled into the hydrolysis (ii) or the washing (iv). By recycling the organic phase comprising organic solvent and DCDPSO and/or DCDPS, the DCDPSO and/or DCDPS is returned into the process and can be gained as product. Thereby, the total yield of DCDPSO and/or DCDPS in the process for producing DCDPSO and/or DCDPS can be increased.

For separating the DCDPSO and/or DCDPS from the polluted water, preferably 0.10 to 5 kg organic solvent per kg polluted water are used. More preferred, 0.15 to 1 kg organic solvent per kg polluted water are used and particularly 0.15 to 0.4 kg organic solvent per kg polluted water.

This amount is sufficient to extract the main part of the DCDPSO and/or DCDPS from the polluted water. After mixing and phase separation, the amount of DCDPSO and/or DCDPS in the water preferably is below 10 ppm. More preferred, after extracting the DCDPSO and/or DCDPS from the polluted water, the water contains less than 5 ppm DCDPSO and/or DCDPS and particularly less than 3 ppm DCDPSO and/or DCDPS. It is an additional advantage, that when using such an amount of organic solvent, the whole organic phase comprising organic solvent and DCDPSO and/or DCDPS can be recycled into the process for producing DCDPSO and/or DCDPS.

To keep the DCDPSO and/or DCDPS in the polluted water in liquid form that it can be removed from the polluted water by mixing with the organic solvent and subsequent phase separation, it is preferred that at least mixing the polluted water with the organic solvent is carried out at a temperature in the range from 10 to 100° C., more preferred at a temperature in the range from 70 to 90° C. and particularly at a temperature in the range from 80 to 90° C. For carrying out the mixing at such a temperature, it is possible to feed the polluted water and the organic solvent with a respective temperature. The polluted water and the organic solvent for example can be heated in storage containers where they are stored before being mixed. For working up the polluted water obtained in the hydrolysis (ii), it is preferred that the temperature at which the mixing is carried out corresponds to the temperature at which the hydrolysis (ii) is carried out. In this case, the polluted water being separated off after the hydrolysis already has the respective temperature and it is only necessary to hold the temperature. This can be achieved for example by insulation of lines through which the polluted water flows and of containers in which the polluted water is stored. However, besides insulation, it is preferred to provide at least the containers in which the polluted water is stored or particularly in case the polluted water directly is fed into the mixing without buffering in a storage container the pipeline through which the polluted water flows with a tempering device to heat or cool the polluted water to the desired temperature. Alternatively, it is also possible to use a separate tempering device, for example a heater, through which the polluted water flows before it is fed into the mixing.

Separating DCDPSO and/or DCDPS from the polluted water by mixing with the organic solvent and subsequent phase separation can be carried out in any suitable apparatus which can be used for a liquid-liquid-extraction process. Suitable apparatus for example are extraction columns like pulsed columns, centrifugal extractors or mixer-settlers. Particularly preferably, extracting is carried out in a mixer-settler. The phase separation into an organic phase and an aqueous phase can be carried out in any suitable phase separator. For supporting phase separation, it is preferred to use a phase separator with internals like knitted fabric or coalescer plates. Further, any suitable measures for supporting phase separation can be performed, for example to increase the phase relationship by adding water or organic solvent or by tilting the mixing device.

To increase the efficiency and thereby to minimize the amount of DCDPSO and/or DCDPS which remains in the water, it is particularly preferred, to carry out mixing and subsequent phase separation in at least two steps and in up to 5 steps. Preferably, the mixing and phase separation is carried out in 1 to 3 steps, particularly in 1 to 2 steps. In each step the polluted water is brought into intense contact with the organic solvent used for extraction. Thereby, it is possible to operate the mixing and phase separation in countercurrent and to feed fresh organic solvent into the last mixing step and to feed the used organic solvent of each step into the previous step. From the first step, the organic solvent enriched in DCDPSO and/or DCDPS then is withdrawn from the mixing and phase separation process. Alternatively, it is also possible to feed fresh organic solvent into each step. However, it is preferred to operate the mixing and phase separation steps in countercurrent.

According to the invention, after carrying out the mixing and subsequent phase separation to remove the DCDPSO and/or DCDPS from the polluted water, the water depleted in DCDPSO and/or DCDPS is subjected to stripping (b) with stripping gas. By stripping (b), particularly organic solvent is removed from the water depleted in DCDPSO and/or DCDPS to allow the organic solvent being reused in the process.

Suitable stripping gases for stripping organic solvent from the aqueous phase for example are inert gases like nitrogen, air or methane, or steam. Particularly preferably, the stripping gas is steam. “Steam” in context of the present invention means water vapor. Preferably, the steam is fresh steam and not produced in an evaporator at the bottom of a stripping column. However, it is also possible to evaporate bottom product of the stripping column and use this evaporated bottom product as stripping gas.

For stripping preferably 0.05 to 0.7 kg steam per kg water depleted in DCDPSO and/or DCDPS are used. More preferred, 0.05 to 0.3 kg steam per kg water depleted in DCDPSO and/or DCDPS are used and particularly 0.05 to 0.15 kg steam per kg water depleted in DCDPSO and/or DCDPS. This amount of steam is sufficient to strip the organic solvent from the water.

The temperature of the water depleted in DCDPSO and/or DCDPS during stripping preferably is in the range from 70 to 150° C. during stripping. By this temperature condensation of the steam used for stripping can be avoided and the organic solvent can be separated from the water depleted in DCDPSO and/or DCDPS by the steam. It is more preferred if the water depleted in DCDPSO and/or DCDPS during stripping has a temperature from 80 to 130° C. and particularly from 100 to 120° C. The steam used for stripping preferably has a temperature at the inlet into the stripping apparatus in the range from 100 to 160° C., more preferred in the range from 110 to 150° C. and particularly in the range from 120 to 145° C.

Stripping preferably is carried out at a pressure in the range from 0.8 to 2.0 bara, more preferred in a range from 0.9 to 1.5 bara and particularly from 1.0 to 1.2 bara.

For stripping, any apparatus suitable for stripping can be used. Particularly, for stripping a stripping column is used. Such a stripping column usually contains internals. Such internals for example are structured packings or random packings or trays. If a random packing is used, the packing for example may contain Raschig®-rings, saddles, Pall@-rings or any other type known to a skilled person. If stripping is carried out in a tray column, any trays can be used by which an intense contact of water depleted in DCDPSO and/or DCDPS and steam can be achieved. Suitable trays for example are perforated trays, bubble cap trays, or valve trays.

If for stripping a tray column is used, the column is operated in counter current, wherein the water depleted in DCDPSO and/or DCDPS is fed at the top of the column and the steam at the bottom of the column. If a column having a structured packing or random packing, a so called packed column, the column can be operated in counter current flow or in co-current flow. However, also when a packed column is used, the column preferably is operated in counter-current.

To achieve a satisfactory result, stripping (b) preferably is carried out in a column with trays as internals and at least two trays. Particularly, the stripping column comprises 5 to 25 trays. Due to the corrosivity of the mixture to be stripped, it is further preferred to coat the interior of the stripping column with enamel or to use a stripping column made of glass.

By stripping the organic solvent can be removed from the water depleted in DCDPSO and/or DCDPS such that after stripping the amount of organic solvent in the water is below 20 ppm, more preferred in a range from 3 to 15 ppm and particularly in a range from 3 to 10 ppm.

For reusing the gaseous components being withdrawn from the stripping, these components preferably are condensed and recycled into the process for producing DCDPSO and/or the process for producing DCDPS. To remove gaseous components which are not condensable, additionally a gas/liquid separation can be provided following the condensation. Such gaseous components for example may be inert gases.

The gaseous component withdrawn from stripping usually is the steam which contains organic solvent which was stripped from the water depleted in DCDPSO and/or DCDPS. After condensing the steam containing the organic solvent, a mixture of water and organic solvent is obtained, which for example can be used as extraction liquid for washing (iv) the organic phase which is obtained in the process for producing DCDPSO.

The water depleted in DCDPSO and/or DCDPS and organic solvent which is obtained as liquid phase after stripping (b) usually will be disposed. If the water depleted in DCDPSO and/or DCDPS and organic solvent contains hydrogen chloride and aluminum chloride, the water will be disposed particularly after neutralizing the hydrogen chloride and aluminum chloride which still is solved in the water by common processes for cleaning the water. By neutralization, usually Al(OH),Clm forms which can be separated off by sedimentation or filtration. After separating off Al(OH),Clm the water can be fed into an activation vessel in a water purification plant. If the water contains alcohols or carboxylic acid, these usually are degraded in a water purification plant.

The mixing and phase separation (a) as well as the stripping (b) can be carried out continuously or batchwise. Preferably, extracting DCDPSO and/or DCDPS from the polluted water by mixing with the organic solvent and subsequent phase separation and stripping organic solvent are carried out continuously. If one of the process steps, either the mixing and phase separation (a) or the stripping (b) is carried out continuously and the other batchwise, it is necessary to provide a buffer container into which the water depleted in DCDPSO and/or DCDPS after mixing and phase separation (a) is collected and to feed the water depleted in DCDPSO and/or DCDPS into the stripping (b) from the buffer container. If the mixing and phase separation (a) is carried out batchwise and the stripping (b) continuously, the buffer container needs to be sufficiently large to receive the whole water depleted in DCDPSO and/or DCDPS which is obtained by the phase separation process. The stripping then is carried out such that from one filling of the container after finishing the extraction to the next filling of the buffer container the contents of the buffer container are fed into the stripping process. If on the other hand the mixing and phase separation (a) is carried out continuously and the stripping (b) batchwise, the container needs to be sufficiently large to collect all of the water depleted in DCDPSO and/or DCDPS which is withdrawn from the phase separation during successional stripping batches. Depending on the amount of polluted water and size of the devices used for mixing and phase separation (a) and stripping (b), it is possible to use only one device or more than one device. If more than one device is used, the devices are connected in parallel to allow simultaneous operation.

An illustrative embodiment of the invention is shown in the figure and explained in more detail in the following description. In the following description, well-known functions or constructions are not described in detail to avoid obscuring the disclosure in unnecessary detail.

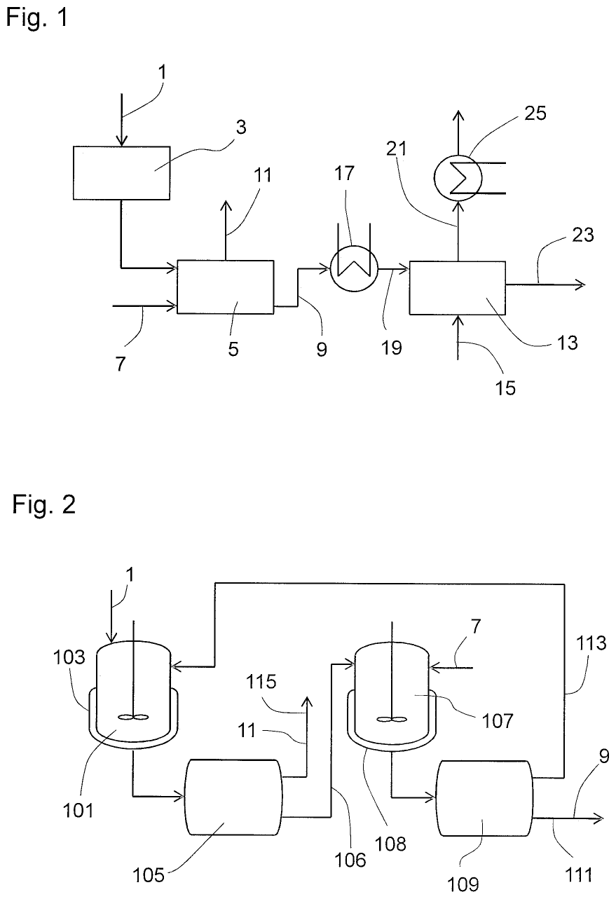

FIG. 1 shows a flow diagram of an embodiment of the inventive process,

FIG. 2 shows a flow diagram of an extraction carried out in two steps,

In the process shown in FIG. 1, polluted water 1 containing DCDPSO and/or DCDPS as impurities is collected in a buffer container 3. From the buffer container, the polluted water 1 is fed into an extraction 5 where the polluted water 1 is mixed with an organic solvent, particularly chlorobenzene, as extractant 7 and subsequently separated into an organic phase and an aqueous phase. In the extraction 5, DCDPSO and/or DCDPS is separated off the polluted water.

The extraction 5 can be carried out in any suitable apparatus for a liquid-liquid extraction. Suitable apparatus for example are columns or mixer-settler. Preferably, the extraction 5 is carried out in a mixer settler. Preferably, the extraction 5 is carried out in at least two steps as shown in FIG. 2, wherein for each step a separate extraction apparatus is used. Thereby, each extraction step can be carried out in a different type of extraction apparatus. However, it is preferred to use only one type of apparatus for each extraction step, particularly a mixer-settler.

The extraction 5 preferably is carried out at a temperature in the range from 70 to 110° C. and at ambient pressure.

From the extraction 5 water depleted in DCDPSO and/or DCDPS 9 and organic solvent enriched in DCDPSO and/or DCDPS 11 are withdrawn. If the organic solvent is chlorobenzene, the organic solvent enriched in DCDPSO and/or DCDPS 11 for example is returned into the production process of DCDPSO, particularly into the hydrolysis of the intermediate reaction product which was obtained by reacting chlorobenzene and thionyl chloride in the presence of aluminum chloride.

The water depleted in DCDPSO and/or DCDPS 9 is subjected to stripping 13. By stripping 13, organic solvent is separated off the water depleted in DCDPSO and/or DCDPS. For stripping 13, steam 15 is brought into contact with the water depleted in DCDPSO and/or DCDPS. Stripping 13 preferably is carried out in a stripping column in which the water depleted in DCDPSO and/or DCDPS and the steam flow in counter current. During stripping the steam is brought into intense contact with the water depleted in DCDPSO and/or DCDPS. Stripping is carried out at a temperature in the range from 80 to 120° C. To achieve this temperature, it is possible to provide a heat exchanger 17 in the line 19 through which the water depleted in DCDPSO and/or DCDPS flows from the extraction 5 to the stripping 13. If the extraction 5 is carried out at the same temperature at which stripping is carried out, it is not necessary to provide the additional heat exchanger 17. In this case it generally is sufficient to insulate the line through which the heated water depleted in DCDPSO and/or DCDPS 9 flows from the extraction 5 to the stripping 13. Alternatively, it is also possible to use the heat of the water depleted in DCDPSO and/or DCDPS 9 for heating the water which is fed into the stripping 13. For using the heat of the water depleted in DCDPSO and/or DCDPS 9 for heating the water which is fed into the stripping 13, any suitable indirect heat exchanger can be used through which the water depleted in DCDPSO and/or DCDPS and the water which is fed into the stripping can flow in separate channels, for example a tube bundle heat exchanger, a plate heat exchanger or a spiral heat exchanger. The water depleted in DCDPSO and/or DCDPS and the water which is fed into the stripping may flow in counter-current flow, co-current flow or cross-flow. Using the heat of the water depleted in DCDPSO and/or DCDPS for heating the water which is fed into the stripping has the additional advantage that the water depleted in DCDPSO and/or DCDPS is cooled.

If an additional buffer container is provided in which the water depleted in DCDPSO and/or DCDPS 9 is collected before being fed into the stripping 13, it is possible to temper the buffer container to heat the water depleted in DCDPSO and/or DCDPS to the temperature at which the stripping 13 is carried out. For tempering, the buffer container may comprise a double jacket or a heating coil through which a heating medium or cooling medium flows or with an electrical heating or with a combination of at least two thereof. A cooling particularly is necessary when the extraction 5 is carried out at a higher temperature than the stripping 13. However, it is preferred to either operate extraction 5 and stripping 13 at the same temperature or stripping 13 at a higher temperature than the extraction 5.

From the stripping, a gaseous stream 21 containing steam and vaporized organic solvent and a liquid stream 23 containing water depleted in DCDPSO and/or DCDPS and organic solvent are obtained. If the organic solvent is chlorobenzene, the gaseous stream 21 preferably is subjected to condensation 25 and then recycled into the production process of DCDPSO, particularly in a washing step of the organic phase obtained by phase separation of the reaction product which is obtained by reacting chlorobenzene and thionyl chloride in the presence of aluminum chloride forming an intermediate reaction product and hydrolysis of the intermediate reaction product in the presence of aqueous hydrogen chloride to form DCDPSO.

The liquid stream 23 obtained by stripping, which contains the water depleted in DCDPSO and/or DCDPS and organic solvent can be withdrawn from the process and introduced in a water purification plant before draining in the environment.

FIG. 2 shows a flow diagram of an extraction process in two steps.

If the extraction 5 is carried out in two steps, the polluted water 1 is fed into a first mixing unit 101. In the first mixing unit 101, the polluted water is mixed with the organic solvent as extractant. The first mixing unit 101 preferably can be heated, for example by a double jacket 103 or a heating coil which is not shown here. The mixture of polluted water and extractant then is fed into a first phase separation unit 105 in which the water and the extractant are separated in an aqueous phase 106 and an organic phase 115.

The first mixing unit 101 and the first phase separation unit 105 preferably are a mixing chamber and a settling chamber of a first mixer-settler.

From the first phase separation unit 105 the aqueous phase 106 is fed into a second mixing unit 107. Further, organic solvent is fed into the second mixing unit 107 as extractant 7. The organic solvent and the aqueous phase are mixed in the second mixing unit 107 and the thus obtained mixture is fed into a second phase separation unit 109. As for the first step, also the second mixing unit 107 preferably can be heated, for example by a double jacket 108 or heating coil which is not shown here. Further, also the second mixing unit 107 and the second phase separation unit 109 preferably are a mixing chamber and a settling chamber of a second mixer-settler. In the second phase separation unit 109, the mixture obtained in the second mixing unit 107 is separated into a second aqueous phase 111 and a second organic phase 113.

The second organic phase 113 is fed into the first mixing unit 101 as extractant. The second aqueous phase 111 is withdrawn from the extraction as water depleted in DCDPSO and/or DCDPS 9 which is fed into the stripping 13.

The organic phase 115 withdrawn from the first phase separation unit 105 is the organic solvent containing DCDPSO and/or DCDPS 11 and is recycled into the production process of DCDPSO if the organic solvent is used in the process for producing DCDPSO.

### EXAMPLES

### Example 1

249 g polluted water withdrawn from the hydrolysis of a process for producing DCDPSO, containing 107 ppm DCDPSO and 25 g chlorobenzene were agitated in a 1-liter vessel at 90° C. and 1000 rpm. After stopping agitating, the mixture separated into an organic phase and an aqueous phase. Samples of the aqueous phase were taken after one minute settling time. 8 ppm DCDPSO were analyzed in the sample.

### Example 2

237 g polluted water withdrawn from the hydrolysis of a process for producing DCDPSO, containing 107 ppm DCDPSO and 49 g chlorobenzene were agitated in a 1-liter vessel at 90° C. and 1000 rpm. After stopping agitating, the mixture separated into an organic phase and an aqueous phase. Samples of the aqueous phase were taken after one minute settling time. 5 ppm DCDPSO were analyzed in the sample. After phase separation, the aqueous phase was mixed again with 40 g chlorobenzene. After stopping mixing, the mixture separated into an organic phase and an aqueous phase. A sample of the aqueous phase taken after 1 minute settling time contained <2 ppm DCDPSO.

### Example 3

In a continuous mixer-settler apparatus 11 kg/h polluted water withdrawn from the hydrolysis of a process for producing DCDPSO, containing 280 ppm DCDPSO were mixed at 80° C. in an agitated vessel of 1 liter volume at 1200 rpm with 1.7 kg/h chlorobenzene and separated in a verti-cal phase separator with a load of 9 m³/(m²h). A sample was taken from the aqueous phase withdrawn from the phase separator. The sample contained 6 ppm DCDPSO.

### Example 4

In a bubble cap column a continuous polluted water stream of 10 kg/h with a chlorobenzene inlet concentration of 802 mg/L and a temperature of 99° C. was stripped with 0.8 kg/h steam, resulting in a steam to polluted water ratio of 0.08 kg/kg. The steam had an inlet temperature of approximately 120° C. The column was operated in counter current mode. To avoid heat losses the column was heated. After stripping, the water depleted in chlorobenzene had a chlorobenzene concentration of 19 mg/L.

1.-17. (canceled)

18. A process for working up water containing 4,4′-dichlorodiphenyl sulfoxide and/or 4,4′-dichlorodiphenyl sulfone as impurities, comprising:
(a) mixing the water containing 4,4′-dichlorodiphenyl sulfoxide and/or 4,4′-dichlorodiphenyl sulfone as impurities with an organic solvent in which 4,4′-dichlorodiphenyl sulfoxide and/or 4,4′-dichlorodiphenyl sulfone have a solubility of at least 0.5 wt % based on the amount of 4,4′-dichlorodiphenyl sulfoxide and/or 4,4′-dichlorodiphenyl sulfone and organic solvent at 20° C., which forms a two-phase system with water and which can be stripped from water with a stripping gas and subsequently separating the obtained mixture into an aqueous phase and an organic phase, and
(b) stripping the organic solvent from the aqueous phase with a stripping gas.

19. The process according to claim 18, wherein the water containing 4,4′-dichlorodiphenyl sulfoxide and/or 4,4′-dichlorodiphenyl sulfone as impurities accrues in a process for producing 4,4′-dichlorodiphenyl sulfoxide and/or 4,4′-dichlorodiphenyl sulfone or in a process using 4,4′-dichlorodiphenyl sulfoxide and/or 4,4′-dichlorodiphenyl sulfone.

20. The process according to claim 18, wherein in (a) 0.10 to 5 kg organic solvent per kg water containing 4,4′-dichlorodiphenyl sulfoxide and/or 4,4′-dichlorodiphenyl sulfone as impurities are used.

21. The process according to a claim 18, wherein the organic solvent is chlorobenzene.

22. The process according to claim 18, wherein the stripping gas is steam.

23. The process according to claim 22, wherein for stripping the organic solvent from the water containing 4,4′-dichlorodiphenyl sulfoxide and/or 4,4′-dichlorodiphenyl sulfone as impurities 0.05 to 0.7 kg steam per kg water containing 4,4′-dichlorodiphenyl sulfoxide and/or 4,4′-dichlorodiphenyl sulfone as impurities are used.

24. The process according to claim 18, wherein the water containing 4,4′-dichlorodiphenyl sulfoxide and/or 4,4′-dichlorodiphenyl sulfone as impurities additionally comprises at least one of chlorobenzene, hydrogen chloride, alkali metal sulfates, metal chlorides, carboxylic acids or alcohols.

25. The process according to claim 24, wherein the amount of chlorobenzene in the water containing 4,4′-dichlorodiphenyl sulfoxide and/or 4,4′-dichlorodiphenyl sulfone as impurities is in the range from 0.01 to 3 wt % based on the total amount of water containing 4,4′-dichlorodiphenyl sulfoxide and/or 4,4′-dichlorodiphenyl sulfone as impurities.

26. The process according to claim 24, wherein the amount of hydrogen chloride in the water containing 4,4′-dichlorodiphenyl sulfoxide and/or 4,4′-dichlorodiphenyl sulfone as impurities is in the range from 1 to 12 wt % based on the total amount of the water containing 4,4′-dichlorodiphenyl sulfoxide and/or 4,4′-dichlorodiphenyl sulfone as impurities.

27. The process according to claim 24, wherein the metal chloride is AlCl₃ and the amount of AlCl₃ in the water containing 4,4′-dichlorodiphenyl sulfoxide and/or 4,4′-dichlorodiphenyl sulfone as impurities is in the range from 10 to 30 wt % based on the total amount of the water containing 4,4′-dichlorodiphenyl sulfoxide and/or 4,4′-dichlorodiphenyl sulfone as impurities.

28. The process according to claim 24, wherein the amount of carboxylic acids in the water containing 4,4′-dichlorodiphenyl sulfoxide and/or 4,4′-dichlorodiphenyl sulfone as impurities is in the range from 1 ppm to 10 wt %.

29. The process according to claim 18, wherein the aqueous phase has a temperature in the range from 70 to 150° C. during stripping.

30. The process according to claim 18, wherein mixing and phase separation (a) is carried out at a temperature in the range from 10 to 100° C.

31. The process according to a claim 18, wherein mixing and phase separation (a) is carried out in at least two steps.

32. The process according to claim 18, wherein after mixing and phase separation, the aqueous phase contains less than 10 ppm 4,4′-dichlorodiphenyl sulfoxide and/or 4,4′-dichlorodiphenyl sulfone.

33. The process according to claim 18, wherein stripping (b) is carried out in a column with at least two theoretical plates.

34. The process according to claim 18, wherein gaseous components being withdrawn from the stripping are condensed and recycled into a process for producing 4,4′-dichlorodiphenyl sulfoxide or into a process for producing 4,4′-dichlorodiphenyl sulfone.
